(19) United States
(12) Patent Application Publication
(10) Pub. No.: US 2009/0171314 A1
(43) Pub. Date: Jul. 2, 2009

(54) MOLDED ARTICLES COMPRISING MICRONEEDLE ARRAYS

(76) Inventor: Dennis E. Ferguson, Mahtomedi, MN (US)

Correspondence Address: 3M INNOVATIVE PROPERTIES COMPANY, PO BOX 33427, ST. PAUL, MN, 55133-3427, US

(21) Appl. No.: 12/295,645

(22) PCT Filed: Apr. 20, 2007
(86) PCT No.: PCT/US07/67026

§ 371 Date: Oct. 1, 2008

Related U.S. Application Data
(60) Provisional application No. 60/793,611, filed on Apr. 20, 2006.

(51) Int. Cl. A61M 5/32; B29C 45/00; B32B 37/00

(52) U.S. Cl. 604/506; 264/328.1; 156/245; 604/239

(57) ABSTRACT

A molded article comprising at least one chain of microneedle arrays wherein adjacent arrays in the chain are interconnected by integrally formed runners. Such a molded article may further comprise two or more chains of microneedle arrays, wherein adjacent chains are interconnected to each other by integrally formed runners. Also, methods of making molded articles and positioning them for delivery to a patient.

### FIELD

The present invention relates to microstructured, molded articles and methods of making the same.

### BACKGROUND

Molded plastic articles are well known and commonly used in everyday life. Most molded articles are relatively large in nature and/or are relatively rugged, and thus may be handled quite conveniently. Certain molded articles, however, are very small and/or include very fine microstructured features, and thus may be difficult to handle conveniently. One example of such articles are arrays of relatively small structures, sometimes referred to as microneedles or micro-pins, which have been disclosed for use in connection with the delivery of therapeutic agents and other substances through the skin and other surfaces. The devices are typically pressed against the skin in an effort to pierce the stratum corneum such that the therapeutic agents and other substances can pass through that layer and into the tissues below.

Devices including fluid passageways in or around the microneedles may be used for delivering a liquid into or through the skin from a reservoir, or alternatively, may be used to draw a liquid from the skin into the device for diagnostic purposes. In one approach, a delivery device comprises an active agent that is externally coated onto an array of microneedles and the active agent is delivered directly into the skin after the microneedles breach the stratum corneum. A number of mechanisms may cause the active agent to be removed from the microneedles and deposited in the skin. For example, the active agent may be directly rubbed off as the microneedles penetrate the skin or the active agent may dissolve off of the microneedles when in contact with interstitial fluid. In certain instances the microneedles may be left in contact with the skin for a specified period of time in order to allow sufficient delivery of the active agent.

### SUMMARY OF THE INVENTION

Molded articles having microstructured features, such as microneedles, are typically quite delicate and may be easily damaged during normal handling. In particular, a number of intermediate handling steps are often necessary to take a molded microstructured article, such as a microneedle array, fashion it into a finished product, and deliver such a product to an end-use customer.

In a first aspect, the present invention is a molded article comprising at least one chain of microneedle arrays wherein adjacent arrays in the chain are interconnected by integrally formed runners. Such a molded article may further comprise two or more chains of microneedle arrays, wherein adjacent chains are interconnected to each other by integrally formed runners.

In a second aspect, the present invention is a method of making a molded article comprising the steps of: (a) providing a mold apparatus comprising an injection gate and a mold insert having the negative image of a plurality of cavities in the form of a chain of arrays interconnected by runners, wherein the mold apparatus has an open position and a closed position; (b) placing the mold apparatus in the closed position; (c) injecting polymeric material through the injection gate into the closed mold apparatus; (d) applying a cavity pack pressure assistance force to each cavity; and (e) opening the mold and removing the molded article from the mold insert.

In a third aspect, the present invention is a microneedle array delivery device comprising a chain of microneedle arrays wherein adjacent arrays in the chain are interconnected by integrally formed runners. The microneedle array delivery device further comprises an application device adapted to receive the chain of arrays, apply a single array to a patient, and advance the chain of arrays so that the next array in the chain is in position for delivery.

As used herein, certain terms will be understood to have the meaning set forth below:

“Microstructure” or “microstructured” refers to specific microscopic features or structures associated with a larger article. By way of example, microstructures can include projections and/or cavities on a surface of a larger article. Such microscopic features will generally have at least one dimension (e.g., length, width, height) that is about 500 microns or less in size.

“Array” refers to medical devices described herein that include one or more structures capable of piercing the stratum corneum to facilitate the transdermal delivery of therapeutic agents or the sampling of fluids through or to the skin.

“Microneedle” or “microarray” refers to specific microscopic structures associated with the array that are capable of piercing the stratum corneum to facilitate the transdermal delivery of therapeutic agents or the sampling of fluids through the skin. By way of example, microneedles can include needle or needle-like structures as well as other structures capable of piercing the stratum corneum.

The features and advantages of the present invention will be understood upon consideration of the detailed description of the preferred embodiment as well as the appended claims. These and other features and advantages of the invention may be described below in connection with various illustrative embodiments of the invention. The above summary of the present invention is not intended to describe each disclosed embodiment or every implementation of the present invention. The Figures and the detailed description which follow more particularly exemplify illustrative embodiments.

### BRIEF DESCRIPTION OF THE DRAWINGS

Preferred embodiments of the invention will now be described in greater detail below with reference to the attached drawings, wherein:

FIG. 1 is a schematic diagram of a molded article.

FIG. 2 is a schematic diagram of another molded article.

FIG. 3 is a schematic diagram of still another molded article.

FIG. 4 is a schematic perspective assembly view of a molding apparatus and a first molded article.

FIG. 5 is a schematic perspective assembly view of a molding apparatus and a second molded article.

While the above-identified drawing figures set forth several embodiments of the invention, other embodiments are also contemplated, as noted in the discussion. In all cases, this disclosure presents the invention by way of representation and not limitation. It should be understood that numerous other modifications and embodiments can be devised by those skilled in the art, which fall within the scope and spirit of the principles of the invention. The figures may not be drawn to scale. Like reference numbers have been used throughout the figures to denote like parts.

### DETAILED DESCRIPTION

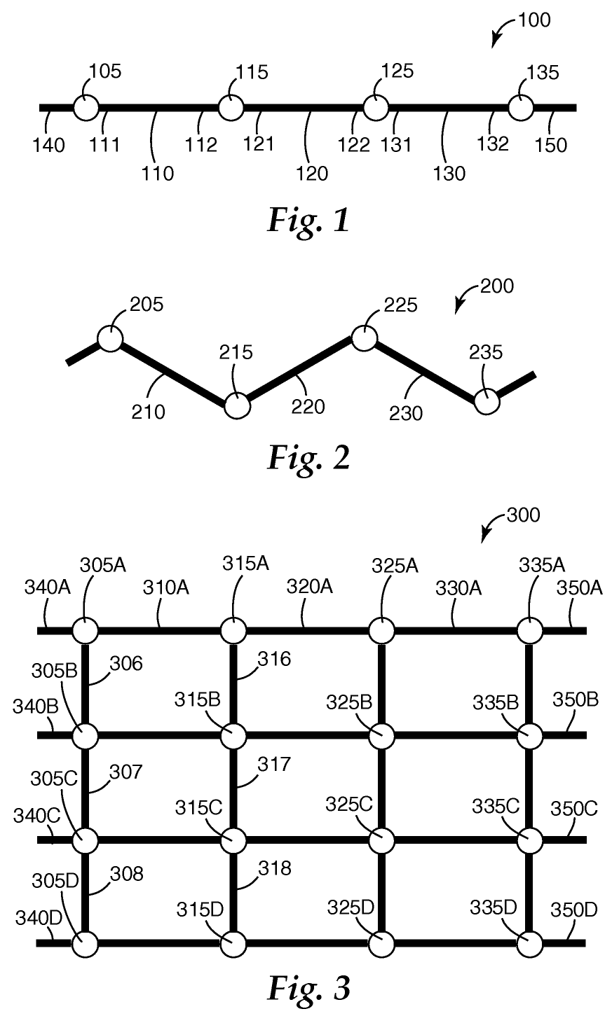

In one embodiment, the molded article of the present invention comprises at least one chain of microneedle arrays. It should be understood that the term “chain” is defined as three or more arrays interconnected by integrally formed runners in a generally linear arrangement. That is, as shown in FIG. 1, a molded article 100 has a first connector (also referred to as a ‘runner’) 110 with a first end 111 and a second end 112 connected to a first array 105 and a second array 115, respectively. A second runner 120 will have a first end 121 and a second end 122 connected to the second array 115 and a third array 125, respectively. Thus a continuous path from the first array 105 to the third array 125 will follow the topological path, first array (105)—first runner (110)—second array (115)—second runner (120)—third array (125). In principle, such an arrangement can be extended indefinitely to form any length chain, for example, to a fourth array 135 connected to the third array 125 through a third runner 130 having first and second ends 131, 132, and so forth. The molded article 100 may have extensions 140, 150 that have a first end connected to an array (e.g., first array 105 and fourth array 135, respectively, as shown in the embodiment of FIG. 1) and a second, “free” end that is not connected to an array. The extensions 140, 150 of the molded article 100 may be useful for a variety of purposes, for example, in aiding handling of the molded article, for providing points of connection to other molded articles, or for aiding in alignment of the molded article in a storage unit or applicator.

The generally linear, or chain-like, arrangement of the arrays interconnected by runners refers to the spatial arrangement of arrays and runners. Although a geometrically linear arrangement as shown in FIG. 1 may be most convenient for certain embodiments, other arrangements may be equally suitable or find specific utility in certain instances. For example, as shown in FIG. 2, the molded article 200 may be in the form of a chain having a zig-zag structure. That is, adjacent runners, such as a first runner 210 and a second runner 220 are oriented at an angle other than 180° with respect to each other. Likewise, the arrays 205, 215, 225, 235 are not arranged along a single geometric line. The third runner 230 is shown oriented along the same axis as the first runner 210, but it should be understood that any regular or random arrangement of interconnecting runners may be employed, including arrangements where the runners are arranged in more than a single plane.

FIG. 3 shows another embodiment where a molded article 300 comprises 4 chains of arrays that are interconnected to each other via integrally formed runners that are aligned generally perpendicular to each chain of arrays. Such an array may be described as forming a 4×4 grid. A first chain of arrays comprises a first runner 310A connected to a first array 305A and a second array 315A, respectively. A second runner 320A connects the second array 315A to a third array 325A, and so forth with a third runner 330A and fourth array 335A. The first chain has extensions 340A and 350A extending from either end of the chain of arrays. A second chain of arrays with arrays 305B, 315B, 325B, 335B is spaced a distance away from and aligned generally parallel to the first chain of arrays. First arrays in each chain 305A-B are interconnected by a runner 306. Second arrays in each chain 315A-B are interconnected by a runner 316, and so forth. Likewise, a third chain of arrays with arrays 305C, 315C, 325C, 335C is spaced a distance away from and aligned generally parallel to the second chain of arrays. First arrays in each chain 305B-C are interconnected by a runner 307. Second arrays in each chain 315B-C are interconnected by a runner 317, and so forth. Such a parallel or grid arrangement of chains of arrays may be extended indefinitely, for example, to a fourth chain of arrays 305D, 315D, 325D, 335D as shown, (connected through runners 308, 318, etc.), and additional chains of arrays (not shown). As shown, all of the chains of arrays have first extensions 340A-D and second extensions 350A-D. Additional extensions (not shown) may be provided extending from arrays on the edges of the grid, for example extending from arrays 315A, 325A, 315D, and/or 325D. In alternative embodiments, one or more of the chains of arrays may have a single extension or no extension. In one embodiment, for example, it may be sufficient for a molded article having several chains of arrays to have only a single extension on either end for handling purposes. As discussed above, such extensions are optional, as for example, a molded article 300 may be handled by the interconnecting runners, thereby providing a more compact total size for the molded article. Such a grid-like structure may be particularly beneficial, in that it may help to provide structural integrity to the molded article 300 thus allowing it to be handled as a single, flat sheet, for example, without an undue amount of bending or warpage.

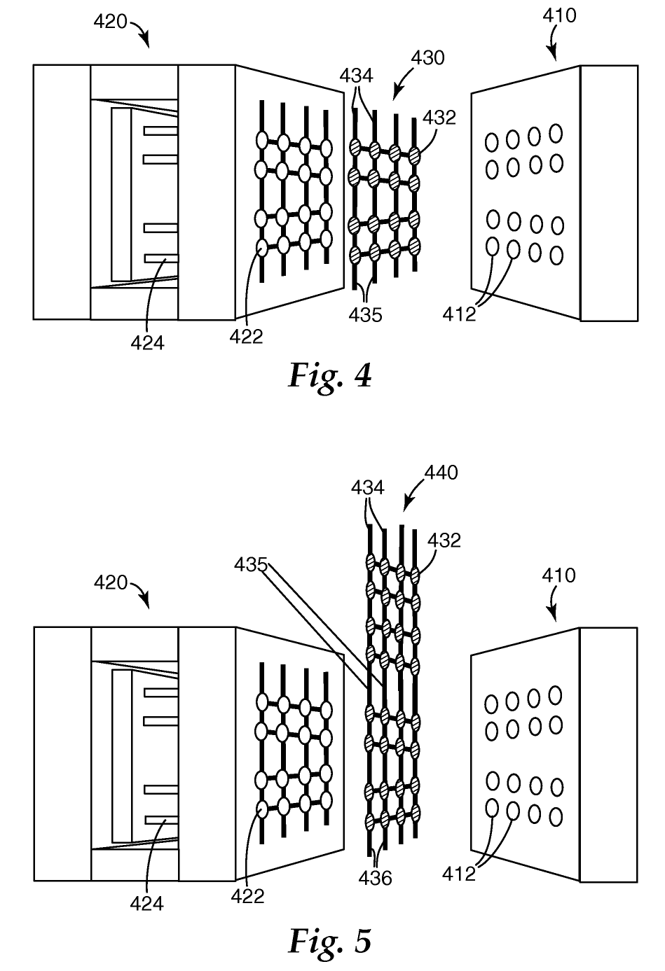

FIG. 4 shows a perspective assembly view of a molding apparatus and a molded article. The molding apparatus comprises a first, or “A”, mold half 410 and a second, or “B”, mold half 420. As shown, each mold half 410, 420 has cavities 412, 422, respectively, that together define the molded shape of a plurality of microneedle arrays 432. The second mold half 420 further has channels that define the molded shape of the runners 434, 435. The molded article 430 is generally prepared by closing the mold (that is, bringing the two mold halves 410, 420 together) and injecting molten polymeric material into the mold through one or more injection ports (not shown), such as a cold or hot sprue, a 3-plate mold, or a hot manifold. The molten material is allowed to cool sufficiently, the mold is opened, and the molded article 430 is removed or ejected from the mold, for example, with the aid of ejector pins 424 in the second mold half 420. The thus formed molded article 430 may then be removed entirely from the molding apparatus for further handling as a 4×4 grid of microneedle arrays. The extending runners 434, 435 may be used for conveniently handling the 4×4 grid without disturbing the delicate microneedles (not shown) on the face of the microneedle arrays 432. In addition to connecting adjacent molded microneedle arrays 432, the runners 434, 435 also serve as overflow vents for each molded microneedle array 432, thus allowing for release of air that might otherwise be trapped in an individual microneedle array cavity within the mold apparatus.

The molded article 430 formed in the process of FIG. 4 may be further processed as shown in FIG. 5 to form a larger molded article 440. After formation of the initial molded article 430 (a 4×4 grid with extensions on two sides), the molded article 430 is partially advanced out of the molding apparatus, so that the microneedle arrays 432 are entirely outside of the molding apparatus, but the extension runners 435 remain within the molding apparatus. The molding apparatus is again closed and molten polymeric material is injected to form a second 4×4 grid of microneedle arrays. The extensions on the leading edge of the second 4×4 grid of microneedle arrays merge with the extensions 435 on the trailing edge of the first 4×4 grid, thereby forming a 4×8 grid of microneedle arrays. The 4×8 grid may be handled by runners 434 and 436 which extend from either side, or these runners may be used to attach to additional chains or grids of arrays to form larger assemblies. This process may be repeated indefinitely, thereby forming chains of microneedle arrays of various lengths to allow for convenient handling of large numbers of delicate microneedle arrays.

One or more replication tools or mold inserts may be placed into a mold apparatus and used to mold polymeric microneedle arrays. In one embodiment, a mold insert may be placed into an injection molding apparatus, molten polymeric material is injected into the molding apparatus under pressure and allowed to fill the mold insert. After the polymeric material is allowed to cool sufficiently, a molded microneedle array is ejected from the molding apparatus. In one aspect, the mold insert may be heated to an elevated temperature prior to injection of the molten polymeric material to aid in filling of the mold insert and subsequently cooled to aid in ejection of the molded part. Further description regarding temperature cycled injection molding may be found in U.S. Pat. No. 5,376,317 (Maus et al.) and International Publication No. WO 05/82596. In another embodiment, a compressive force may be used to assist during an injection molding process. Further description regarding this so-called injection-compression molding may be found in U.S. Pat. Nos. 4,489,033 (Uda et al.), 4,515,543 (Hamner), and 6,248,281 (Abe et al.), and U.S. Patent Application Ser. No. 60/634,319 filed on Dec. 7, 2004. In addition, ultrasonic energy may be used to assist in filling of the mold insert with molten polymeric material, as described in U.S. Patent Application Ser. No. 60/634,319 filed on Dec. 7, 2004. The disclosures of all of the foregoing molding patents are herein incorporated by reference.

A wide variety of polymeric materials may be suitable for use in molding microneedle arrays. In one embodiment, the material is selected so that it is capable of forming relatively rigid and tough microneedles that resist bending or breaking when applied to a skin surface. In one aspect, the polymeric material has a melt-flow index greater than about 5 g/10 minutes when measured by ASTM D1238 at conditions of 300° C. and 1.2 kg weight. The melt-flow index is often greater than or equal to about 10 g/10 minutes and sometimes greater than or equal to about 20 g/10 minutes. In another embodiment, the tensile elongation at break as measured by ASTM D638 (2.0 in/minute) is greater than about 100 percent. In still another embodiment, the impact strength as measured by ASTM D256, “Notched Izod”, (73° F.) is greater than about 5 ft-lb/inches. Examples of suitable materials include polycarbonate, polyetherimide, polyethylene terephthalate, and mixtures thereof. In one embodiment the material is polycarbonate.

A chain of microneedle arrays may be further handled while interconnected. For example, the chain may be used as a means to transport arrays to a separate coating station where a pharmaceutical preparation is applied to the surface of the needles. If such a preparation is applied with use of a carrier fluid that is subsequently allowed to evaporate, then the chain may further serve to transport the array from the coating station to a drying station, such as an oven. The chain with attached arrays may also be used to transport arrays to a converting station where additional components, such as a skin facing adhesive may be added to the array. The chain of arrays may be stored for later use or processing (e.g., with the aid of a covering surface or liner to protect the integrity of the microneedles).

In one embodiment, a chain of microneedle arrays may be used directly in an application device. For example, a dispenser could store a large number of microneedle arrays in a chain and feed the arrays individually to a dispensing or application port that would separate an individual array from the chain and apply it to a patient. Alternatively, the arrays may be separated and packaged individually after undergoing any subsequent processing desired as described above.

In one embodiment, the microneedle arrays may be used to make patches having a flexible backing with a skin-contacting pressure-sensitive adhesive suitable for adhering the patch to a skin surface. Such a microneedle patch may be prepared by adhering an adhesive patch (i.e., a backing film with an adhesive layer on one surface) to the back or non-structured side of an array, so that the adhesive patch extends beyond the perimeter of the microneedle array. The microneedles in an array may be arranged in any desired pattern or distributed over the substrate surface randomly. In one embodiment, the microneedles are arranged in uniformly spaced rows placed in a rectangular arrangement. In one embodiment, the area having microneedles on the patient-facing surface of a device is more than about 0.1 cm² and less than about 20 cm², and in some instances more than about 0.5 cm² and less than about 5 cm². The microneedles are typically less than 1000 microns in height, often less than 500 microns in height, and sometimes less than 250 microns in height. The microneedles are typically more than 5 microns in height, often more than 25 microns in height, and sometimes more than 100 microns in height.

The microneedles may be characterized by an aspect ratio. As used herein, the term “aspect ratio” is the ratio of the height of the microneedle (above the surface surrounding the base of the microneedle) to the maximum base dimension, that is, the longest straight-line dimension that the base occupies (on the surface occupied by the base of the microneedle). In the case of a pyramidal microneedle with a rectangular base, the maximum base dimension would be the diagonal line connecting opposed corners across the base. Microneedles typically have an aspect ratio of between about 2:1 to about 5:1 and sometimes between about 2.5:1 to about 4:1.

The microneedle arrays prepared according to any of the foregoing embodiments may comprise any of a variety of configurations, such as those described in the following patents and patent applications, the disclosures of which are herein incorporated by reference. One embodiment for the microneedle devices comprises the structures disclosed in U.S. Patent Application Publication No. 2003/0045837. The disclosed microstructures in the aforementioned patent application are in the form of microneedles having tapered structures that include at least one channel formed in the outside surface of each microneedle. The microneedles may have bases that are elongated in one direction. The channels in microneedles with elongated bases may extend from one of the ends of the elongated bases towards the tips of the microneedles. The channels formed along the sides of the microneedles may optionally be terminated short of the tips of the microneedles. The microneedle arrays may also include conduit structures formed on the surface of the substrate on which the microneedle array is located. The channels in the microneedles may be in fluid communication with the conduit structures. Another embodiment for the microneedle devices comprises the structures disclosed in co-pending U.S. Patent Application Publication No. 2005/0261631 which describes microneedles having a truncated tapered shape and a controlled aspect ratio. Still another embodiment for the microneedle arrays comprises the structures disclosed in U.S. Pat. No. 6,313,612 (Sherman, et al.) which describes tapered structures having a hollow central channel. Still another embodiment for the microneedle arrays comprises the structures disclosed in U.S. Pat. No. 6,379,324 (Gartstein, et al.) which describes hollow microneedles having at least one longitudinal blade at the top surface of tip of the microneedle.

One manner in which the microneedles of the present invention may be characterized is by height as measured from a substrate surface. It may be preferred, for example, that the base-to-tip height of the microneedles be about 500 micrometers or less as measured from the substrate surface. Alternatively, it may be preferred that the height of the microneedles is about 250 micrometers or less as measured from the base to the tip. It may also be preferred that the height of molded microneedles is greater than about 90%, and more preferably greater than about 95%, of the height of the cavities in the mold insert that are negative images of the desired microneedle shape. The microneedles may deform slightly or elongate upon ejection from the mold insert. This condition is most pronounced if the molded material has not cooled below its softening temperature, but may still occur even after the material is cooled below its softening temperature. It is preferred that the height of the molded microneedles is less than about 115%, and more preferably less than about 105%, of the height of the cavities in the mold insert that are negative images of the desired microneedle shape.

The general shape of the microneedles of the present invention may be tapered. For example, the microneedles may have a larger base at the substrate surface and extend away from the substrate surface, tapering to a tip. In one embodiment the shape of the microneedles is pyramidal. In another embodiment, the shape of the microneedles is generally conical. In one embodiment the microneedles have a defined tip bluntness, such as that described in co-pending and commonly owned U.S. Patent Application Publication No. 2005/0261631, wherein the microneedles have a flat tip comprising a surface area measured in a plane aligned with the base of about 20 square micrometers or more and 100 square micrometers or less. In one embodiment, the surface area of the flat tip will be measured as the cross-sectional area measured in a plane aligned with the base, the plane being located at a distance of 0.98 h from the base, where h is the height of the microneedle above the substrate surface measured from base to tip. The microneedles may have shafts with a variety of shapes, for example, a pyramid, cone, or blade, as well as a bases with a variety of shapes, for example, a square, rectangle, or oval.

In one embodiment, the negative image(s) of the at least one microneedle is substantially completely filled with injected polymeric material prior to opening the mold and ejecting the part. By substantially completely filled, it should be understood that the molded microneedle should have a height greater than about 90 percent of the corresponding height of the microneedle topography in the mold insert. In one embodiment, the molded microneedle has a height greater than about 95 percent of the corresponding height of the microneedle topography in the mold insert. It is preferable that the molded microneedle has a height substantially the same (e.g., 95 percent to 105 percent) as the corresponding height of the microneedle topography in the mold insert.

Mold inserts suitable for use in the present invention may be made by any known conventional method. In one method, a positive ‘master’ is used to form the mold insert. The positive master is made by forming a material into a shape in which the microneedle array will be molded. This master can be machined from materials that include, but are not limited to, copper, steel, aluminum, brass, and other heavy metals. The master can also be made from thermoplastic or thermoset polymers that are compression formed using silicone molds. The master is fabricated to directly replicate the microneedle array that is desired. The positive master may be prepared by a number of methods and may have microneedles of any of a variety of shapes, for example, pyramids, cones, or pins. The protrusions of the positive master are sized and spaced appropriately, such that the microneedle arrays formed during molding using the subsequently formed mold insert have substantially the same topography as the positive master.

A positive master may be prepared by direct machining techniques such as diamond turning, disclosed in U.S. Pat. No. 5,152,917 (Pieper, et al.) and U.S. Pat. No. 6,076,248 (Hoopman, et al.), the disclosures of which are herein incorporated by reference. A microneedle array can be formed in a metal surface, for example, by use of a diamond turning machine, from which is produced a mold insert having an array of cavity shapes. The metal positive master can be manufactured by diamond turning to leave the desired shapes in a metal surface which is amenable to diamond turning, such as aluminum, copper or bronze, and then nickel plating the grooved surface to provide the metal master. A mold insert made of metal can be fabricated from the positive master by electroforming. These techniques are further described in U.S. Pat. No. 6,021,559 (Smith), the disclosure of which is herein incorporated by reference.

Microneedle arrays prepared by methods of the present invention may be suitable for delivering drugs (including any pharmacological agent or agents) through the skin in a variation on transdermal delivery, or to the skin for intradermal or topical treatment, such as vaccination.

In one aspect, drugs that are of a large molecular weight may be delivered transdermally. Increasing molecular weight of a drug typically causes a decrease in unassisted transdermal delivery. Microneedle devices suitable for use in the present invention have utility for the delivery of large molecules that are ordinarily difficult to deliver by passive transdermal delivery. Examples of such large molecules include proteins, peptides, nucleotide sequences, monoclonal antibodies, DNA vaccines, polysaccharides, such as heparin, and antibiotics, such as ceftriaxone.

In another aspect, microneedle arrays prepared by methods of the present invention may have utility for enhancing or allowing transdermal delivery of small molecules that are otherwise difficult or impossible to deliver by passive transdermal delivery. Examples of such molecules include salt forms; ionic molecules, such as bisphosphonates, preferably sodium alendronate or pamedronate; and molecules with physicochemical properties that are not conducive to passive transdermal delivery.

In another aspect, microneedle arrays prepared by methods of the present invention may have utility for enhancing delivery of molecules to the skin, such as in dermatological treatments, vaccine delivery, or in enhancing immune response of vaccine adjuvants. Examples of suitable vaccines include flu vaccine, Lyme disease vaccine, rabies vaccine, measles vaccine, mumps vaccine, chicken pox vaccine, small pox vaccine, hepatitis vaccine, pertussis vaccine, rubella vaccine, diphtheria vaccine, encephalitis vaccine, yellow fever vaccine, recombinant protein vaccine, DNA vaccine, polio vaccine, therapeutic cancer vaccine, herpes vaccine, pneumococcal vaccine, meningitis vaccine, whooping cough vaccine, tetanus vaccine, typhoid fever vaccine, cholera vaccine, tuberculosis vaccine, and combinations thereof. The term “vaccine” thus includes, without limitation, antigens in the forms of proteins, polysaccarides, oligosaccarides, or weakened or killed viruses. Additional examples of suitable vaccines and vaccine adjuvants are described in United States Patent Application Publication No. 2004/0049150, the disclosure of which is hereby incorporated by reference.

Microneedle devices may be used for immediate delivery, that is where they are applied and immediately removed from the application site, or they may be left in place for an extended time, which may range from a few minutes to as long as 1 week. In one aspect, an extended time of delivery may be from 1 to 30 minutes to allow for more complete delivery of a drug than can be obtained upon application and immediate removal. In another aspect, an extended time of delivery may be from 4 hours to 1 week to provide for a sustained release of drug. In one aspect, the drug may be applied to the skin (e.g., in the form of a solution that is swabbed on the skin surface or as a cream that is rubbed into the skin surface) prior to applying the microneedle device.

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow.

We claim:

1. A molded article comprising at least one chain of microneedle arrays wherein adjacent arrays in the chain are interconnected by integrally formed runners.

2. The molded article as claimed in claim 1 comprising 8 or more arrays.

3. The molded article as claimed in claim 1 comprising 20 or more arrays.

4. The molded article as claimed in claim 1, comprising two or more chains of microneedle arrays, wherein adjacent chains are interconnected to each other by integrally formed runners.

5. The molded article as claimed in claim 4 wherein at least one runner connecting adjacent chains is integrally formed with one array on each chain.

6. The molded article as claimed in claim 4 wherein at least one runner connecting adjacent chains is integrally formed with a runner on each chain.

7. The molded article as claimed in claim 1 wherein the microneedle arrays comprise at least one microneedle with a base-to-tip height of about 500 μm or less.

8. The molded article as claimed in claim 1 wherein the microneedle arrays comprise a polymeric material.

9. The molded article as claimed in claim 8 wherein the polymeric material is polycarbonate.

10. A method of making a molded article comprising the steps of:
(a) providing a mold apparatus comprising an injection gate and a mold insert having the negative image of a plurality of cavities in the form of a chain of arrays interconnected by runners, wherein the mold apparatus has an open position and a closed position;
(b) placing the mold apparatus in the closed position;
(c) injecting polymeric material through the injection gate into the closed mold apparatus;
(d) applying a cavity pack pressure assistance force to each cavity; and
(e) opening the mold apparatus and removing the molded article from the mold insert.

11. The method as claimed in claim 10 wherein the molded article comprises a chain of microneedle arrays wherein adjacent arrays in the chain are interconnected by integrally formed runners.

12. The method as claimed in claim 11 and further comprising the steps of:
(f) advancing the chain of microneedle arrays so that all of the arrays are outside of the mold apparatus and so that one of the integrally formed runners remains within the mold apparatus; and
(g) repeating steps (b) to (e), thereby forming a second chain of microneedle arrays integrally connected to the first chain.

13. A method of making a microneedle array comprising the steps of:
(a) preparing a molded article according to the method of claim 11; and
(b) separating the microneedle array from the chain of arrays by severing all of the runners interconnected to the microneedle array being separated.

14. A method of making a microneedle array patch comprising the steps of:
(a) preparing a molded article according to the method of claim 11;
(b) adhering an adhesive patch to one microneedle array; and
(c) separating the microneedle array from the chain of microneedle arrays by severing all of the runners interconnected to the microneedle array being separated.

15. The method as claimed in claim 13 wherein each runner is separated at a junction between the runner and the array.

16. A microneedle array delivery device comprising:
a chain of microneedle arrays wherein adjacent arrays in the chain are interconnected by integrally formed runners, and an application device adapted to receive the chain of arrays.

17. The microneedle array delivery device as claimed in claim 16 wherein the application device is further adapted to apply a single microneedle array to a patient and advance the chain of arrays so that the next array in the chain is in position for delivery.

18. A method of applying a microneedle array to a mammal comprising the steps of:
(a) providing the microneedle array delivery device as claimed in claim 17;
(b) positioning the device proximate to a skin surface;
(c) applying the single microneedle array from the chain of microneedle arrays to the skin surface; and
(d) advancing the chain of arrays so that the next array in the chain is in position for application.
